(19) United States
(12) Patent Application Publication
(10) Pub. No.: US 2018/0020186 A1
(43) Pub. Date: Jan. 18, 2018

(54) SYSTEMS AND METHODS FOR INTEGRATION OF AUDIOVISUAL COMPONENTS TO CENTRAL STATION MONITORING

(71) Applicant: University of Maryland Medical Center, Baltimore, MD (US)

(72) Inventor: Samuel Tefera GURMU, Baltimore, MD (US)

(21) Appl. No.: 15/635,765

(22) Filed: Jun. 28, 2017

Related U.S. Application Data
(60) Provisional application No. 62/355,807, filed on Jun. 28, 2016.

(51) Int. Cl. H04N 7/10; H04N 5/38; G06T 3/40; H04N 5/44; A61B 5/00

(52) U.S. Cl. CPC H04N 7/104; H04N 5/44; A61B 5/7425; G06T 3/40; H04N 5/38

(57) ABSTRACT

Systems and methods for integration of audiovisual components of central station monitoring are provided. At the outset, patient data from a central monitoring station is received at an audiovisual switcher. The patient data is from a plurality of patient monitors, each patient monitor corresponding to a respective patient of a plurality of patients. The patient data from the central monitoring station is continuously transmitted to an integrated terminal that is communicatively coupled to the audiovisual switcher. The patient data is also selectively transmitted to a user terminal that is communicatively coupled to the audiovisual switcher.

### PRIORITY

This application claims the benefits of U.S. Provisional Application No. 62/355,807, filed on Jun. 28, 2016, which is incorporated herein by reference in its entirety.

### FIELD OF INVENTION

The present invention generally relates to systems and methods for patient monitoring, and more particularly, for integrating audiovisual components of central station monitoring.

### BACKGROUND

When treating a patient, physicians, nurses, and other medical professionals rely on a variety of devices to assist with the monitoring of the patient during treatment. In hospitals and other healthcare or clinical settings, a variety of patient vital signs may be continuously monitored. Example vital signs include pulse rate, breathing rate, blood oxygen level, blood pressure, and others. Continuous monitoring of various physiological conditions of the patient may be achieved with a patient monitoring system. The information gathered through the continuous monitoring of the patient is used to assist medical professionals in providing healthcare to the patient. For example, the patient monitoring system assists healthcare providers at the point of care. In other example, the patient monitoring system monitors conditions during on-going medical procedures.

Until now, and as discussed below, it is difficult to monitor multiple patients simultaneously. Accordingly, the inventor has provided an improved patient monitoring system.

### SUMMARY OF INVENTION

Accordingly, the embodiments of the present invention are directed to systems and methods for integration of audiovisual components of central station monitoring that substantially obviate one or more problems due to limitations and disadvantages of the related art.

In one example embodiment, the systems and methods for integration of audiovisual components of central station monitoring may include one or more input ports configured to receive patient data from the central monitoring station, one or more output ports configured to continuously transmit the patient data from the central monitoring station to an integrated terminal that is communicatively coupled to the audiovisual switcher, and one or more output ports configured to selectively transmit the patient data from the central monitoring station to a user terminal that is communicatively coupled to the audiovisual switcher.

In another example embodiment, the systems and methods for integration of audiovisual components of central station monitoring may include receiving, at an audiovisual switcher, patient data from the central monitoring station, continuously transmitting, at the audiovisual switcher, the patient data from the central monitoring station to an integrated terminal that is communicatively coupled to the audiovisual switcher, and selectively transmitting, at the audiovisual switcher, the patient data from the central monitoring station to a user terminal that is communicatively coupled to the audiovisual switcher.

In the various example embodiments, other features include that the patient data received from the central monitoring station is retrieved from a plurality of patient monitors, each patient monitor corresponding to a respective patient of a plurality of patients, the central monitoring station is one of a plurality of central monitoring stations, each central monitoring station being configured to monitor a plurality of patients, the patient data for the plurality of central monitoring stations are simultaneously displayed in a matrix format, the patient data for one of the plurality of central monitoring stations is selectively displayed in a matrix format, patient data that is continuously transmitted incudes patient data in a matrix format corresponding to a plurality of patients, the patient data that is selectively transmitted incudes only patient data for a single patient, the audiovisual switcher includes a video processing unit having a scaling unit, one or more input ports of the audiovisual switcher enables the central monitoring station to be controlled remotely, and the patient data that is continuously transmitted is displayed on a first display unit, and data that is selectively transmitted is displayed on a second display unit.

Additional features and advantages of the invention will be set forth in the description which follows, and in part will be apparent from the description, or may be learned by practice of the invention. The advantages of the embodiments of the present invention will be realized and attained by the structure particularly pointed out in the written description as well as the appended drawings.

It is to be understood that both the foregoing general description and the following detailed description are examples and are intended to provide further explanation of the invention.

### BRIEF DESCRIPTION OF THE DRAWINGS

Further embodiments, details, advantages, and modifications will become apparent from the following detailed description of the preferred embodiments, which is to be taken in conjunction with the accompanying drawings.

FIG. 1 illustrates a schematic view of a patient monitoring system according to an example embodiment of the present invention.

FIG. 2 illustrates a primary central monitoring station display and a secondary central monitoring display according to an example embodiment of the present invention.

FIG. 3 illustrates a flow diagram of functionality for integration of audiovisual components of central station monitoring according to an example embodiment of the present invention.

### DETAILED DESCRIPTION OF THE INVENTION

The embodiments of the present invention generally relate to systems and methods for integrating audiovisual components of central station monitoring, and more particularly, systems and methods for integrating audiovisual components for continuous physiological monitoring of patients and reviewing patient data without disrupting the display of live, continuous, and multi-patient physiological data.

Primary components may include bedside or portable patient monitors which collect data from each patient, a central monitoring station which collects and stores information received from multiple patient monitors, and one or more displays that graphically render the patients' data as a matrix of individual windows. Here, each window may correspond to an individual patient. The central monitoring station has multiple video outputs that include a primary output to continuously display the matrix and a secondary output that graphically renders detailed patient data for a single patient in response to a user request (i.e., one of the patients depicted on the primary display).

In conventional systems, the visualization of a single patient's data poses several significant drawbacks. When selecting to view a single patient's data, a common practice referred to as “full disclosure work” on a single display configuration (i.e., single video signal), each display associated with the central monitoring station necessarily removes the multi-patient matrix from the view in favor of a single patient view. This allows for the review of past physiological data for the single patient and is frequently necessary when administering clinical care. However, when single patient reviews occur, patient alarms on that central monitoring station are only audible at the station. In addition, indicators of the source of the alarms and the alarm condition are much obscured.

In such conventional systems, secondary displays (i.e., multiple video outputs) are not used and all display terminals visualize the single patient review taking place at the central monitoring station. In other words, all displays showing multiple patients and live alarms become obscured by a single medical professional reviewing a single patient's historical data. In additional, larger clinical units require multiple central monitoring stations to adequately handle the patient capacity. Thus, in order to investigate full disclosure work for a specific patient, the clinician needs to be at the specific central monitoring station to which that patient's monitors are connected. In clinical settings, medical professionals need to visualize the physiological data from multiple locations in a patient care area. Often, video splitters are used to replicate the display in other areas, but without the ability to remotely control the display content.

In addition, work areas where medical professionals frequently work are not typically located next to the locations of central monitoring stations. As a result, additional stations are needed at those locations to mirror central monitoring stations in a 1:1 manner. These spaces require the dedication of a single work area for this purpose in settings where space is in high demand and expensive.

Integration of additional information that may aid in clinical workflow and/or notifications has not been available through patient monitoring systems. Patient monitoring systems are closed/proprietary systems that are heavily protected by the manufacturers. Enabling general computer use through a central monitoring station is very difficult.

Reference will now be made in detail to the embodiments, examples of which are illustrated in the accompanying drawings. In the following detailed description, numerous specific details are set forth in order to provide a thorough understanding of the present invention. However, it will be apparent to one of ordinary skill in the art that the present invention may be practiced without these specific details. In other instances, well-known methods, procedures, components, and circuits have not been described in detail so as not to unnecessarily obscure aspects of the embodiments. Wherever possible, like reference numbers will be used for like elements.

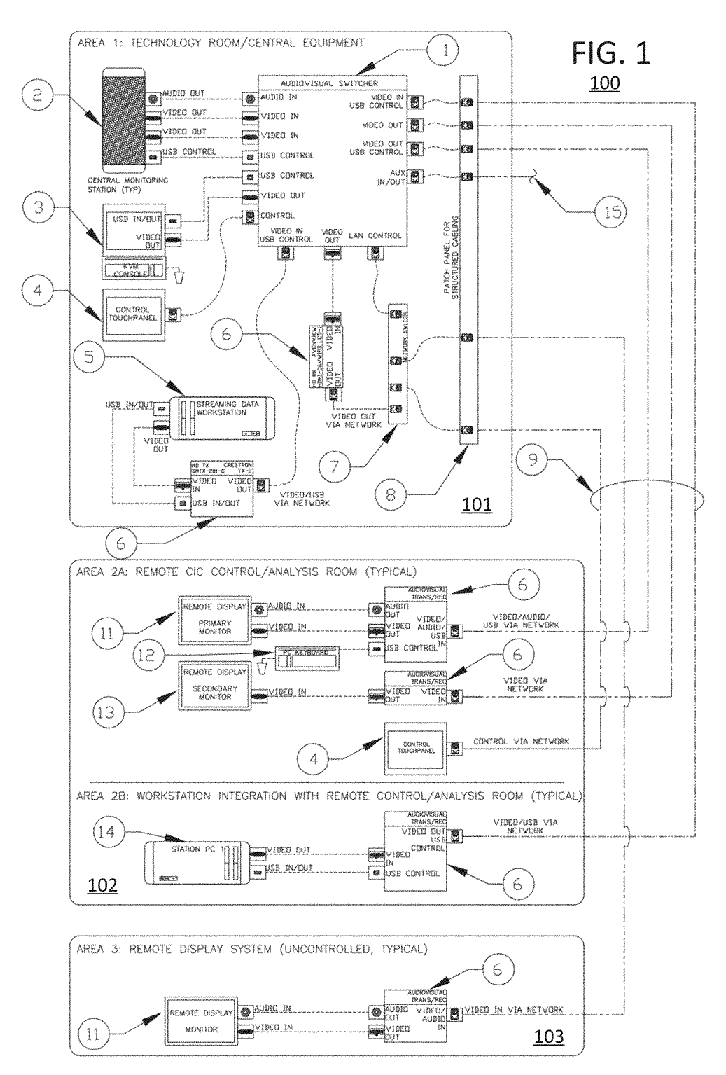

FIG. 1 illustrates a schematic view of a patient monitoring system 100 according to an example embodiment of the present invention. As shown in FIG. 1, patient monitoring system 100 may include central equipment 101, controller equipment 102, and remote display equipment 103. It should be readily understood by one of ordinary skill in the art that the configuration depicted in FIG. 1 is an example, and that other configurations are feasible without departing from the scope of the embodiments of the invention.

Central equipment 101 may include audiovisual switcher 1, one or more central monitoring stations 2, user console 3 (e.g., a keyboard, mouse, audio output, video output, etc.), system interface control 4 (e.g., touch panel controller), content delivery workstation 5, one or more audiovisual transceivers 6 (i.e., transmit and receive), network switch 7, and structured cabling patch panel/cross-connect 8. The various components of central equipment 100 may be located as part of a technology room for a patient care area or any area where centralized equipment is located.

As shown in FIG. 1, audiovisual switcher 1 includes a plurality of inputs configured to receive a variety of input types. For example, the input types of audiovisual switcher 1 include known and expected audio and video formats, as well as universal serial bus (“USB”) control, etc. Audiovisual switcher 1 is configured to receive multiple inputs from central monitoring stations 2, user console 3, system interface control 4, content delivery workstation 5, and audiovisual transceivers 6. Input signals may be directly or indirectly coupled to audiovisual switcher 1. For example, the respective outputs of central monitoring stations 2, user console 3, and system interface control 4 may be directly coupled to audiovisual switcher 1. In another example, the output of content delivery workstation 5 may be indirectly supplied to audiovisual switcher 1 through audiovisual transceivers 6.

In addition, audiovisual switcher 1 includes a plurality of outputs configured to transmit a variety of output types. The output types of audiovisual switcher 1 include audio, video, universal serial bus (“USB”) control, local area network (“LAN”) control, auxiliary outputs, etc. The output ports of audiovisual switcher 1 are respectively coupled to audiovisual transceivers 6, network switch 7, and structured cabling patch panel/cross-connect 8.

Audiovisual switcher 1 is a device designed to receive and transmit digital audiovisual (“AV”) content from multiple inputs to multiple outputs as programmed into the switcher. Input/output sources may be of various digital formats. Audiovisual switcher 1 may be fixed or modular in design. Audiovisual switcher 1 may be a single unit or multiple units integrated to function as a single unit or as multiple units with cross-unit communications and routing. In some embodiments, audiovisual switcher 1 may include one or more video processing units, such as a scaling unit, to ensure that high resolution displays are provided according to display size or type.

Central monitoring station 2 is a device attached to a patient monitoring system that serves as the central point of collection for patient data from physiological monitors and ancillary equipment feeding into monitors. For example, this device may monitor 8-32 patients depending on manufacturer and configuration.

User console 3 (e.g., a keyboard, mouse, audio output, video output, etc.) is an input/output device that enables keyboard, mouse, audio, and video functionality to a device connected to audiovisual switcher 1 or to the user console directly.

System interface control 4 is a device that enables selection and control of other devices attached to audiovisual switcher 1 for such functions as defining audiovisual input/output functionality.

Content delivery workstation 5 is a device that provides digital content to audiovisual switcher 1 for use in display overlays, distinct sub-windows on displays, or full display information. Content delivery workstation 5 also may function to input other network-based or direct-attached devices to audiovisual switcher 1.

Audiovisual transceiver 6 is a device that may accept audiovisual output (e.g., video, audio, control, etc.) and convert/transmit those signals (e.g., analog, digital) over a structured cable plant 9. Audiovisual transceiver 6 may receive digital signals through structured cable plant 9 and may convert/transmit those signals to audiovisual input (e.g., video, audio, control, etc.). Audiovisual transceiver 6 may include transmitter functionality, receiver functionality, or both. Connections may be through any audiovisual connector type to equipment with audiovisual ports. Connections to structured cable plant 9 may be by any connector required based on structured cable plant 9, including the use of wireless segments within that cable plant.

Network switch 7 is a device that enables other devices connected to it to exchange information through any known or expected network protocols used by the connected devices and understood by the network switch.

Structured cabling patch panel/cross-connect 8 is a passive device at a central location used to terminate horizontal network cables extended throughout an area. Structured cabling patch panel/cross-connect 8 enables an additional cable, attached to one horizontal cable at the patch panel, to extended communications from a distant device to a device at the central location (e.g., network switch 7, or audiovisual switcher 1).

Structured cable plant 9 is an infrastructure of cabling used to connect distant devices to a central location (e.g., based on linetype). Included in structured cable plant 9 (e.g., information and communications technology (“ICT”)) may be segments utilizing wireless communication devices to connect distant devices to network switch 7.

Other local connectivity (not shown) based on other (e.g., proprietary, based on linetype, etc.) communication types may be used. For example, a connection between devices utilizing cabling not part of structured cable plant 9. This may include, but not be limited to, video cables (e.g., DVI, HDMI, etc.), audio cables (e.g., ⅛″ jacks, rc-style cables), and control cables (e.g., USB cables, rs232 cables, etc.). The connection may utilize wireless communications between devices (e.g., Bluetooth, zigbee, etc.).

Peripheral integration 15 (e.g., cameras, etc.) is any device with a digital or analog (typically converted to digital) signal that may provide input to audiovisual switcher 1 indirectly via an audiovisual transceiver 6 or directly via structure cable plant 9 and an auxiliary port of audiovisual switch 1. In addition, devices for peripheral integration 15 may communicate with audiovisual switcher 1 through network switch 7.

In some embodiments, central monitoring stations 2 may be located remotely and communicatively coupled to audiovisual switcher 1, such as through structured cable plant 9 utilizing audiovisual transceivers 6 (configuration not shown in FIG. 1). Similarly, content delivery workstation 5 may be located remotely and communicatively coupled to the audiovisual switcher 1 through structured cable plant 9 utilizing audiovisual transceiver 6 or through network communications to audiovisual switcher 1 (configuration not shown in FIG. 1).

As discussed above, patient monitoring system 100 includes central equipment 101, controller equipment 102, and remote display equipment 103. Central equipment 101 may be located in a technology room or central equipment location, illustrated as area 1. Controller equipment 102 may be located in remote locations where there is user interaction with the central equipment 101. Controller equipment 102 may include a core area illustrated as area 2a with optional equipment illustrated by area 2b. Remote display equipment 103 is equipment that may be located in remote display areas, illustrated as area 3, with no direct user interaction with central equipment 101.

Controller equipment 102 includes a plurality of audiovisual transceivers 6, each of audiovisual transceivers 6 being coupled to central monitoring station display 11, secondary central monitoring station display 13, and workstation 14 (e.g., a personal computer, laptop, or tablet), respectively. In an example embodiment, central monitoring station display 11 is an integrated or a primary display configured to continuously display patient data in a matrix format, as described above. Secondary central monitoring station display 13 may display patient data in a matrix format, but also may display more detailed patient data (e.g., for a single patient) in response to a request of a medical professional user. In addition, controller equipment 102 may include remote keyboard/mouse 12 and system interface control 4 (e.g., a touch panel controller).

Central monitoring station display 11 is a primary display that graphically renders the output from audiovisual switcher 1 as defined by the programming/settings of audiovisual switcher 1. For example, displayed are the outputs for multiple patients from one or more central monitoring stations 2 in a matrix format.

Remote keyboard/mouse 12 is an input/output device, such as a keyboard or mouse, which is configured to control a remote device through audiovisual switcher 1. For example, remote keyboard/mouse 12 may control the central monitoring station 2.

Secondary central monitoring station display 13 is a secondary display that graphically renders the output from audiovisual switcher 1 as defined by the programming/settings of audiovisual switcher 1. For example, displayed are the outputs or detailed patient data of a single patient from the central monitoring station 2.

Workstation 14 is a computer workstation configured to utilize the display of central monitoring station 2 for its own use.

At remote locations where user-interaction is needed, such as controller equipment 102, devices utilize the structured cable plant 9 to connect directly to audiovisual switcher 1. Input/output ports of audiovisual switcher 1 are connected through structured cabling patch panel/cross-connect 8 to audiovisual transceivers 6 to primary and secondary central monitoring station displays 11, 13. A basic keyboard/mouse 12 may connect to audiovisual transceiver 6 that is configured to send control signals to audiovisual switcher 1. The Internet-based system interface control 4 enables the user to select the device controlled by keyboard/mouse 12 and the patient data visualized on the primary and secondary central monitoring station displays 11, 13. As multiple central monitoring stations 2 may be coupled to audiovisual switcher 1, system interface control 4 enables the user to select a specific group of patients to visualize and to enable full disclosure work to take place. Video and USB connectivity are routed through audiovisual transceivers 6 to audiovisual switcher 1. This enables a common keyboard/mouse 12 and video display to be switched between central monitoring stations 2 with its functionality and the workstation 14 with its pc-based functionality.

Similar to controller equipment 102, remote display equipment 103 includes one or more audiovisual transceivers 6, each of the audiovisual transceivers 6 being coupled to central monitoring station display 11. Audiovisual transceiver 6 enables communication to central equipment 101 through structured cable plant 9. The remote locations without user-interaction functionality enable users to visualize patient data from central monitoring station 2.

At remote locations where control is not needed, such as remote equipment 103, a single central monitoring station display 11 connects through audiovisual transceiver 6. Audiovisual transceiver 6 is network-based and directly connected to network switch 7. In addition, network switch 7 is directly connected to audiovisual switcher 1. This enables any location with access to structured cable plant 9 to connect to central monitoring stations 2. In typical configurations, interactive devices are not installed at the locations of remote equipment 103 as they are typically in corridors, nursing alcoves, and staff spaces.

By using audiovisual components to manage the video output from central monitoring station 2, the audio and/or visual data may be managed and distributed to resolve the aforementioned drawbacks of conventional systems. Multiple central monitoring stations 2 may be connected to a single audiovisual switcher 1 enabling audio and/or visual data to be routed to any work area or user terminal communicatively connected to audiovisual switcher 1. Remote displays, such as primary central monitoring station display 11 and/or secondary central monitoring station display 13, and remote work area workstations, such as workstation 14, may be connected to audiovisual switcher 1 using known and expected data networks to enable medical professionals to have full access to patient data from any location.

By connecting the video outputs from central monitoring stations 2 to audiovisual switcher 1, both primary (e.g., patient matrix) and secondary outputs (e.g., individual patient data) may be routed to various locations. The outputs to specific dual-screen work areas may be routed by audiovisual switcher 1. Primary displays are always available to show current patient information including alarm states and visual notifications. The secondary display at all connected work areas enables the medical professional to conduct full disclosure work on the secondary display without disrupting the live patient matrix. This reduces risk of delays in responding to alarms as location and condition are always available on unit displays. Using audiovisual switcher 1 enables this work to take place at any location accessible across the network.

In additional, by using system interface control 4, a medical professional may select which central monitoring station 2 is accessed when working in a large unit with multiple central monitoring stations 2. In some configurations, access to specific central monitoring stations 2 may be limited to particular medical professionals (e.g., one at a time), however, such configurations allow for an unlimited number of work areas to be configured for this access. It also allows a single work area to have access to any central monitoring station 2 throughout the medical facility that is connected to audiovisual switcher 1. For example, a local workstation (e.g., personal computer, laptop, or tablet) for general office use may be routed through audiovisual switcher 1 enabling the screens or dual screens at the work area to be used for general office use. This workstation may be at the work area or in a centralized area.

The use of audiovisual switcher 1 enables any video content to be integrated into the displays. This information may be derived from a content delivery workstation with input from automated systems or through manual input from integrated devices communicating with the workstation. In additional, direct-connect and network-based devices may input content to audiovisual switcher 1 for display as programmed by audiovisual switcher 1.

Central monitoring stations 2 enable multiple (e.g., two or more) video outputs. The primary output is configured to display the combined information for all patients connected to central monitoring station 2 in a matrix format. The secondary output is further configured to display a single patient's information while enabling the primary display to retain the contents of all patients in the matrix. Rather than displaying these outputs on local displays, the output is supplied to the audiovisual switcher 1. To enable control of central monitoring station 2, the USB port of central monitoring station 2 may be connected directly to audiovisual switcher 1. By connecting the video outputs and USB control through audiovisual switcher 1, other devices connected to audiovisual switcher 1 have access to the video output and USB control. To augment content to the patient matrix or to a single patient display, a content delivery workstation 5 may be connected to the audiovisual station 1. Additional augmentation is possible through the use of network-based or other direct-connect peripheral integration 15 (e.g., cameras, etc.). Any digital content may be visualized once attached to audiovisual switcher 1. Augmented content may be visualized as banners, running tickets, custom matrix layouts, and the like on primary central monitoring station display 11 and/or secondary central monitoring station display 13.

The various components of patient monitoring system 100 may include one or more processors, codecs, memory devices, communication devices, and the like. For example, a communication device (e.g., such as audiovisual transceiver 6) may be configured to transmit and/or receive patient data from remote sources. In another example, the communication device may include a network interface card that is configured to provide wired or wireless network communications. A variety of wireless communication techniques may be used including infrared, radio, Bluetooth, Wi-Fi, and/or cellular communications. Alternatively, the communication device may be configured to provide wired network connection(s), such as an Ethernet connection.

One or more processors may include general or specific purpose processors to perform computation and control functions of patient monitoring system 100. Processors may include a single integrated circuit, such as a micro-processing device, or may include multiple integrated circuit devices and/or circuit boards working in cooperation to accomplish the functions of the processor.

Memory devices may contain various components for retrieving, presenting, modifying, and storing data. Memory devices may include a variety of computer-readable media. In the various embodiments, memory devices may include volatile and nonvolatile medium, removable and non-removable medium. For example, memory devices may include any combination of random access memory (“RAM”), dynamic RAM (DRAM), static RAM (SRAM), read only memory (“ROM”), flash memory, cache memory, and/or any other type of non-transitory computer-readable medium.

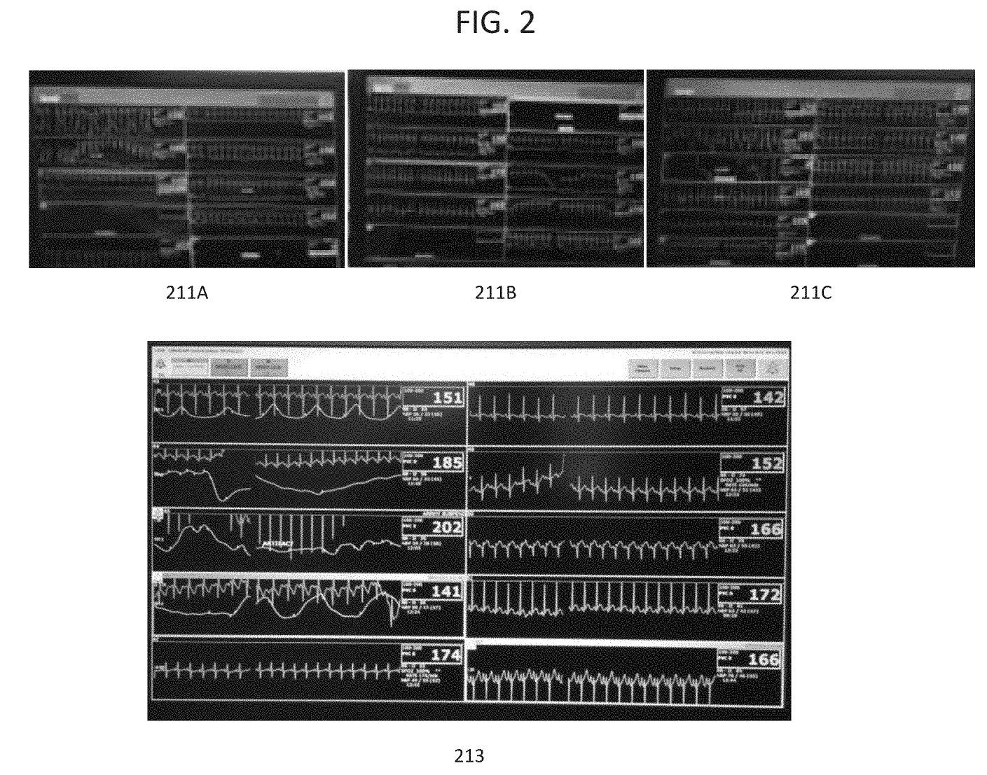

FIG. 2 illustrates a primary central monitoring station display 211 and a secondary central monitoring display 213 according to an example embodiment of the present invention.

As shown in FIG. 2, primary central monitoring station display 211 includes primary central monitoring station displays 211A, 211B, and 211C. Each of primary central monitoring station displays 211A, 211B, and 211C may correspond to a respective central monitoring station (such central monitoring station 2 of FIG. 1) of a plurality of central monitoring stations. Primary central monitoring station display 211 is configured to provide continuous physiological monitoring of patients and reviewing patient data without disrupting the display of live, continuous, and multi-patient physiological data (i.e., matrix data). By contrast, secondary central monitoring display 213 is further configured to graphically render detailed patient data for a single patient in response to a user request. For example, the medical professional may select to view detailed patient data from among the patients shown in the matrix.

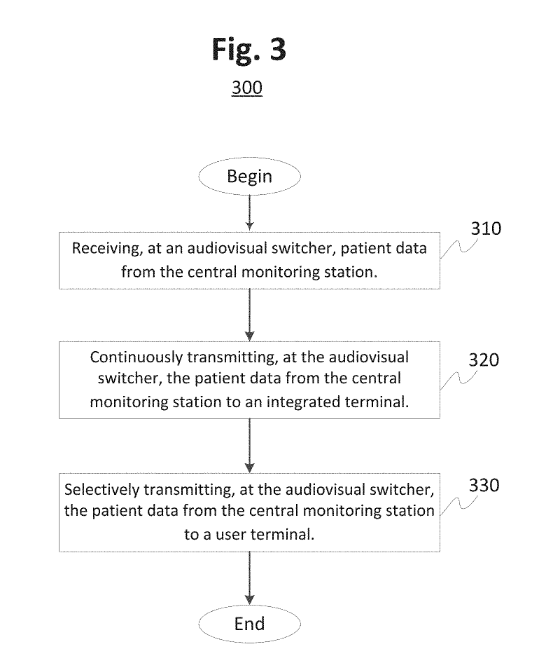

FIG. 3 illustrates a flow diagram of a functionality 300 for integration of audiovisual components of central station monitoring according to an example embodiment of the present invention. In some instances, the functionality of the flow diagram of FIG. 3 is implemented by software stored in memory or other computer readable or tangible media, and executed by a processor. In other instances, the functionality may be performed by hardware (e.g., through the use of an application specific integrated circuit (“ASIC”), a programmable gate array (“PGA”), a field programmable gate array (“FPGA”), etc.), or any combination of hardware and software.

At 310, functionality 300, receives, from a central monitoring station, patient data from a plurality of patient monitors. Each patient monitor may correspond to a respective patient of a plurality of patients. Next, at 320, functionality 300 continuously transmits the patient data from the central monitoring station to an integrated terminal that is communicatively coupled to the audiovisual switcher. Lastly, at 330, functionality 300 selectively transmits the patient data the central monitoring station to a user terminal that is communicatively coupled to the audiovisual switcher.

As discussed above, the various embodiments may include the audiovisual switcher that enables passage of audiovisual and control signals between the patient monitoring system and a plurality of end points, the console that enables the control of a plurality of control monitoring stations from a single input source, the workstation that enables the input of controlled content for display with data from the patient monitoring system, the input component that enables additional content to be displayed with the data from the patient monitoring platform as integrated by the audiovisual switcher, the audiovisual transceiver that enable the connection of various audiovisual equipment to the audiovisual switcher so as to extend patient monitoring data to remote locations in controlled and in uncontrolled conditions, the system interface controls that enable patient monitoring system data to be selectively displayed as well as bypassed in favor of local workstations, and a dual-screen display configuration that secures the display of critical patient monitoring information at all times while allowing for analysts of patient data on secondary displays.

Thus, the addition of audiovisual switching to a patient monitoring system augments the functionality of the system in ways beyond the current design and functionality of conventional patient monitoring systems. It will be apparent to those skilled in the art that various modifications and variations may be made in the systems and methods for integration of audiovisual components to central station monitoring of the present invention without departing from the spirit or scope of the invention.

I claim:

1. An audiovisual switcher, comprising:
one or more input ports configured to receive patient data from the central monitoring station;
one or more output ports configured to continuously transmit the patient data from the central monitoring station to an integrated terminal that is communicatively coupled to the audiovisual switcher; and
one or more output ports configured to selectively transmit the patient data from the central monitoring station to a user terminal that is communicatively coupled to the audiovisual switcher.

2. The audiovisual switcher according to claim 1, wherein the patient data received from the central monitoring station is retrieved from a plurality of patient monitors, each patient monitor corresponding to a respective patient of a plurality of patients.

3. The audiovisual switcher according to claim 1, wherein the central monitoring station is one of a plurality of central monitoring stations, each central monitoring station being configured to monitor a plurality of patients.

4. The audiovisual switcher according to claim 3, wherein patient data for the plurality of central monitoring stations are simultaneously displayed in a matrix format.

5. The audiovisual switcher according to claim 3, wherein patient data for one of the plurality of central monitoring stations is selectively displayed in a matrix format.

6. The audiovisual switcher according to claim 1, wherein the patient data that is continuously transmitted incudes patient data in a matrix format corresponding to a plurality of patients.

7. The audiovisual switcher according to claim 1, wherein the patient data that is selectively transmitted incudes only patient data for a single patient.

8. The audiovisual switcher according to claim 1, wherein the audiovisual switcher includes a video processing unit having a scaling unit.

9. The audiovisual switcher according to claim 1, wherein one or more input ports of the audiovisual switcher enables the central monitoring station to be controlled remotely.

10. The audiovisual switcher according to claim 1, wherein patient data that is continuously transmitted is displayed on a first display unit, and data that is selectively transmitted is displayed on a second display unit.

11. A method for integration of audiovisual components of a central monitoring station, the method comprising:
receiving, at an audiovisual switcher, patient data from the central monitoring station;
continuously transmitting, at the audiovisual switcher, the patient data from the central monitoring station to an integrated terminal that is communicatively coupled to the audiovisual switcher; and
selectively transmitting, at the audiovisual switcher, the patient data from the central monitoring station to a user terminal that is communicatively coupled to the audiovisual switcher.

12. The method according to claim 11, wherein the patient data received from the central monitoring station is retrieved from a plurality of patient monitors, each patient monitor corresponding to a respective patient of a plurality of patients.

13. The method according to claim 11, wherein the central monitoring station is one of a plurality of central monitoring stations, each central monitoring station being configured to monitor a plurality of patients.

14. The method according to claim 13, wherein patient data for the plurality of central monitoring stations are simultaneously displayed in a matrix format.

15. The method according to claim 13, wherein patient data for one of the plurality of central monitoring stations is selectively displayed in a matrix format.

16. The method according to claim 11, wherein the patient data that is continuously transmitted incudes patient data in a matrix format corresponding to a plurality of patients.

17. The method according to claim 11, wherein the patient data that is selectively transmitted incudes only patient data for a single patient.

18. The method according to claim 11, wherein the audiovisual switcher includes a video processing unit having a scaling unit.

19. The method according to claim 11, wherein one or more input ports of the audiovisual switcher enables the central monitoring station to be controlled remotely.

20. The method according to claim 11, wherein patient data that is continuously transmitted is displayed on a first display unit, and data that is selectively transmitted is displayed on a second display unit.
